(19) United States
(12) Patent Application Publication
(10) Pub. No.: US 2021/0308294 A1
(43) Pub. Date: Oct. 7, 2021

(54) SYSTEM PERFORMING INACTIVATION OF MICROORGANISMS ON PAPER NOTES IN ATM WITH PULSED LIGHT TECHNOLOGY

(71) Applicant: MONEYSHOWER TEKNOLOJI ARASTIRMA GELISTIRME SANAYI VE TICARET ANONIM SIRKETI, Maltepe (TR)

(72) Inventors: FURKAN ERUÇAN, Sariyer (TR); KÜBRA KARAKOÇ, Muratpasa (TR)

(21) Appl. No.: 16/852,704

(22) Filed: Apr. 20, 2020

(30) Foreign Application Priority Data
Apr. 2, 2020 (TR) 2020/05270

(51) Int. Cl. A61L 2/10

(52) U.S. Cl. CPC A61L 2/10; G07F 19/201

(57) ABSTRACT

The invention relates to a system which disinfects the microorganisms on the banknotes in ATM (automatic teller machine) machines. The invention is characterized by the application of the pulsed lights system (1) seen in FIG. 1 to the ATM (2) for the disinfection of the banknotes in it as can be seen in FIG. 2 and FIG. 3.

### TECHNICAL FIELD

The invention relates to a system which disinfects the microorganisms on the banknotes in ATM (automatic teller machine) machines.

### STATE OF THE ART

People are usually informed and warned by the doctors with the help of media especially in winters against the risk of epidemics spread by paper notes. People try to solve this problem by washing their hands with a disinfecting chemical after they touch the paper note since a current technology that can disinfect the paper note is not available. In case people clean their hands with chemical disinfectant substances so as not to be exposed to the microorganisms causing disease that will contaminate from money, this will lead to an increase in exposure to chemicals and mostly it is not possible to reach these chemicals immediately after contact with money. Moreover, States try to solve this problem by means of burning paper notes in determined periods of time. For example, the Central Bank in the presence of the Turkish Banks Association often calls for banks to collect the dirty money in circulation and to deliver the same to them so as to eliminate the epidemics spread through the paper notes. Particularly in the swine influenza in the year 2009, banknote branches were established in Turkey in 7 regions and the monies in the circulation were collected and burned in these branches. Frequently burning the banknotes in circulation in the banknote branches by the states has many disadvantages. Firstly, this system does not have an environmentalist and sustainable approach. Contrary to what is believed, the paper notes are not manufactured from paper but they are manufactured from the mixture of cotton, linen and other specific chemicals. Every country has many hidden technological steps while printing their money so as to prevent the counterfeit money. In a research made, it is found that the average life of the high currencies is 15 years while the average life of low currencies is 6 years. Therefore, withdrawal of the money from circulation and burning the same and reprinting the same before the material of the money completes its economic life is both a non-economic and an non-environmental friendly solution. Additionally, the banknotes are withdrawn from circulation regardless of their microbial load on them at the phase of collecting the dirty banknotes in circulation. As a result of the studies made, it was found out that, there is an average of 2400 bacteria on the banknote even they look new. It is study which was made with the cooperation of MasterCard and Oxford University in the year 2013. In this study, there are 26000 bacteria on the banknotes; on the other hand there are 2400 bacteria on the cleanest and newest notes.

In the past, 3 different methods were used for the disinfection of paper materials. These are heat, gas and ultraviolet rays. Disinfection with heat leads to the deformation of the material of the money and shortens the circulation period of money. The disinfection module with gas has not been actualized due to the fact that the gas mixture which is used consists of gases that are harmful to human health. Disinfection module with the ultraviolet rays is supported by a fan and it is transformed into a module which can be integrated into the money cassettes in ATM. The method is completely different. In this module, the problem was tried to be solved with an additional module of the pusher arm and with high constant air flow of the ventilator system given from the sideways and short edges of the banknotes within the money cassettes where the banknotes in bundles are compressed by the pusher arm. The banknotes are enabled to be compressed only from their central points by means of a part to be attached on the pusher arm and the banknotes are aimed to remain free except the single point where they are compressed. It is aimed to provide an access of beams for making disinfection between the free banknotes by means of the air inlet exiting out of the ventilator system. Therefore, it is possible to perform disinfection during minimum 15 minutes in the ATMs where 90% of the real money flow is realized. In the new system that we proposed, disinfection is integrated to different sections in the ATM in a path-breaking manner and instant disinfection of the banknote is provided by means of using “pulsed light” as a novel method.

Studies for disinfection of solid materials with heat, gas and ultraviolet rays have been made in the past. Disinfection process with heat damages the material of the paper note and the gas which is used for killing the microorganisms carries a great risk for human health. Some of the studies made with the ultraviolet rays are based on single and long-term disinfection of paper notes and this disinfection model could not be applicable in ATM. In another ultraviolet beam model, a device which performs disinfection by ultraviolet ray by separating the money bundles with the ventilator within the money cassettes where the paper notes are stored in the ATM. The disinfection duration with this device is minimum 15 minutes. It is not possible to achieve high disinfection efficiency with this system. At the same time, decrease in the cassette volume due to the product to be located within the money cassettes and decrease of the money capacity that the cassette can receive are the other negative features. Some of the studies made are for producing new ATM models. This is a condition which is costly and time consuming.

The money is disinfected for minimum 15 minutes in the fan assisted UV-C beam system which is a system that aims to solve this problem in ATMs where the physical money flow is mostly realized. In fact, it is not possible to apply this system in Recycler ATMs which were started to be sold in the year 2017 and were purchased by many banks to replace the current ATMs. Since the money deposited by a customer in this model ATMs is placed in front of the money cassette, the next customer can withdraw this money that is located to the front from the ATM. In this case, disinfection time for 15 minutes is insufficient since it is a very long time to wait in front of ATM for a customer. In this fan assisted disinfection module, the parts of the system (motor etc.) are placed inside the money cassette and it decreases the amount of paper note that the cassette can receive, increases the frequency of the paper note storage in the ATM and causes extra personnel cost. (3 employees are assigned for the operations that require service, maintenance-repair of ATM, opening ATM for money storage.)

The most efficient factor in the creation of the invention is that the pathogens (causing disease) of bacteria and viruses in every environment on earth lead to contagious diseases; they play an efficient role in carrying these bacteria and viruses. The paper notes which touch average 150 different human hands are contaminated within 1 month after each touch and are transferred to the next person after they are handled by people who are in different physical activities from different professions (1). Many studies were made until today in terms of the bacteriological inspection of paper notes; however no solid solution proposal has been presented.

The patent numbered CN101114392 is only a method and does not propose a solution or an expression in terms of the applications to ATMs. In said invention, a separate box in which the paper note in ATM is required to enter. This process makes it necessary to manufacture a new ATM.

The patent numbered CN202216021 disclosed that LED light can be used in the sterilisation method of bacteria and virus instead of sterilisation with gas. However, the system is not a development of a system for disinfecting the monies within the ATM; also in this study it does not propose a solution in terms of its integration to paper notes or ATMs.

The patent numbered JP2007172566 is related to the requirement of making a separate device for the sterilisation of microorganism and to their usage in the houses. In said patent, it is stated that it is required to place two separate money depots in the ATM and it is required to sterilize the money in one depot and to send it to the other depot.

The patent numbered US2011253563A1 relates to that it is required to make a new ATM model for the sterilisation of microorganism and bacteria. In this document, it is aimed to carry out the disinfection process of the paper note and coins together by using pressurized explosion system by inventing a separate drawer. It is not stated how it is integrated into the ATMs.

In the patent numbered CN103977439A, plasma technology is used, it is not stated for ATMs and it is directed for developing a completely different product for the sterilisation of money and there is no statement in terms of its ATM integration.

In the patent numbered CN101789090A, again the fan system is used as a dust absorber and it is placed in a separate manner and various mechanical equipment like fan etc. are mentioned.

In the patent numbered WO2016190828, there is a disinfection process performed on only the touch screen and keyboard of the ATM by the needle tip spray with the pump system, here the external section of the ATM is disinfected. There is no application for the disinfection of the paper notes by providing change in the cartridges in ATM.

### DEFINITION OF THE INVENTION

The present invention is related to system which is developed in order to eliminate the abovementioned disadvantages and to bring new advantages to the relevant technical field.

The aim of the invention is to create an environmentalist ATM integrated to all current ATMs. Design and production of a novel ATM model is long-term and costly process. The other studies aim to purify the microorganisms from the paper note and to realize this within a manufactured separate unit. On the other hand in this system, it is aimed to disinfect the paper notes in the ATM by means of applying the “pulsed light” to the band, belt paths enabling the progress of the paper notes within the ATM and paper note exit area from the money cassette. It bears a novel invention signature with its features that it has an integration area within the ATM and it is a method which is new for the paper note disinfection within the ATM not used before in any ATMs in the world. The present ATMs used today are transformed into an environmentalist ATMs with this technology.

The main aim of the invention is to disinfect all paper notes returned to ATM by means of destroying microorganisms on the paper noted and to decrease the drug consumption which is imported to our country. Additionally, the paper notes with cleaned surface facilitate the money identification of ATMs and shorten the duration of this process. This invention is a system which has a mechanical and electrical design integrated to the distributor, pic module (selection module) within the ATM. This could be realized by means of exposing the money that flows on the band or belt to intensive light. Here the application to be pulseded is the integration of the system which results from particularly developed heavy R&D for the distributor or selection module within the ATM system and here there is no condition threatening the inventive step. The intensive light system cannot be able to penetrate into the solid substance; it can only be used for surface cleaning. For this reason, its use is during the outlet of the money from the selection module or during the money transfer on the band or belt. Therefore, each paper note will be exposed to surface disinfection. The interaction of the user with the microorganisms causing disease will be minimized. As it can be seen during the last few years, contagious diseases have become a great problem which spread enormously and threaten the lives of people. One of the most important factors in spread of this thread is based on not providing the disinfection of the paper notes that frequently pass from hand to hand in a regular manner. This integrated system with completed R&D will have an important role in the selection of this invention by the banks for showing that they are valuing their target customer population and at the same time they aim to be better than their competitors. Since there are no substitutes for this in the world and at the same time there is no similar system in Turkey and in the world, this can be considered as an important factor. The main aim of our invention is to kill the microbiological and pathogenic (causing disease) livings on the paper notes, namely to purify the paper from dirt and disinfect the same. In this manner, in the conditions of today, the diseases that contaminate by means of money which can contact the whole society will be substantially eliminated in a tangible manner.

Although the microbes come to our body through the paper notes do not affect the human body immediately, since these microorganisms will continue to be present in the human body, they will show their effect in case they lose their immunity. If we require describing this with a simple example, a person suffering from cold can experience diseases such as typhoid, paratyphoid or dysentery. Our aim is to eliminate these diseases and subsequently to reduce the costs of the doctor, medicine, hospital costs.

### DRAWINGS

The embodiments of the present invention which are above summarized in brief and discussed in detail herein below can be understood by means of referring to the exemplary embodiments of the invention which are illustrated in the attached drawings. However it shall be noted that the attached drawings hereto only illustrate the typical embodiments of this invention, and for this reason this invention may permit other equally effective embodiments, it shall be mentioned that these are not considered as limiting the scope of the invention.

FIG. 1 is a schematic view of the pulsed UV beam technology.

FIG. 2 is a side view of the ATM money chamber where the invention is applied.

FIG. 3 is a perspective view of the ATM money chamber where the invention is applied.

FIG. 4 is a perspective view of the ATM money chamber showing the application of the invention.

In order to facilitate understanding and to mention the common equivalent elements in the drawings, identical reference numbers are used where appropriate. The figures are not scaled and they are simplified for clarity. It is considered that the elements and characteristics of one embodiment can be incorporated into other embodiments in an beneficial manner without requiring additional explanation.

### DESCRIPTION OF THE DETAILS IN THE DRAWINGS

The equivalents of the reference numbers shown in the figures are given herein below.
1—Pulsed light system
2—ATM
3—Distributer
4—Cassette outlet
5—Upper selection module
6—Lower selection module
7—Belt
8—Band
9—Money inlet
10—Power Source
11—Storage capacitor
12—Inductor
13—Light source
14—Intensive beam
15—Target
16—Money cassette

### DETAILED DESCRIPTION OF THE INVENTION

In this detailed description, the preferred alternatives of the inventive structure are described only for clarifying the subject matter such that no limiting effect is created.

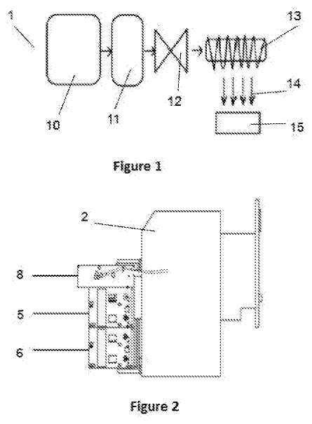

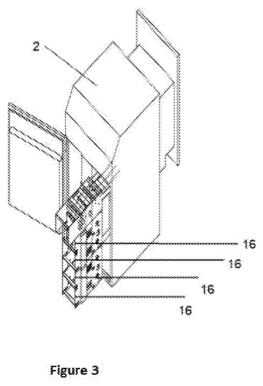

The invention is characterized by the application of the pulsed lights system (1) seen in FIG. 1 to the ATM (2) for the disinfection of the banknotes in it as can be seen in FIG. 2 and FIG. 3.

In the invention as seen in FIG. 1, the pulsed light system (1) is given for producing intensive beam (14) to the light source (13) by taking to the inductor (12) by means of storing within the storage capacitor (11) of the received energy fri a power source (10). The generated UV-C beam with disinfection feature is intensified and applied to the target (15). Pulsed light (PL) is a technique which decontaminates the surfaces by killing the microorganisms by means of using intense wide-spectrum pulses which are rich in terms of UV-C beam. The pulsed light technology comprises usage of short term intensive pulses and a wide spectrum for enabling microbial inactivation on the surface of any kind of material. The electromagnetic energy accumulates in a capacity during a certain period of time and then it emits in the form of light within a short period of time, this leads to an increase in power with minimum additional energy consumption.

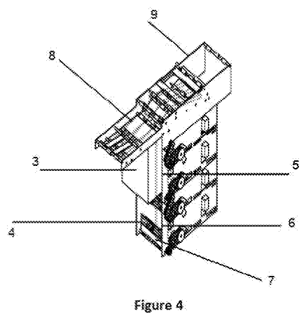

In the invention, in FIG. 4, how the pulsed light system (1) is used within the ATM (2) in money transfer process is shown. Typically, the pulsed light system (1) can be positioned on at least one of the following; one or more adjustable light source (13) units, the upper and lower selection modules (5, 6), the level of the cassette outlet (4) points, belt system (7) and band (8) system or money inlet (9) sections. In addition to this, in the invention, the pulsed light system (1) light source (13) can be positioned within the money cassettes (16) as in FIG. 3. A power source (10) can be positioned in empty spaces within the ATM (2), for example in the empty spaces on the sides of the upper and lower selection modules (5, 6). The light generated by means of the light source (13) can comprise wide spectrum wavelengths from UV to infrared.

The money that exit from the money cassettes (16) is transferred to the housing at the beginning of the belt (7) and band (8) system and they are collected here and then it advances to the outlet entrance by means of the band system (8) individually or collectively according to the model of the ATM (2). The disinfection with the pulsed light is provided in the path followed by the money as of its exit from the money cassette (16) to its leaving the ATM (2) within the money cassettes (16) of the ATM (2) with the pulsed light system (1), or provided by applying the combinations of these.

In the invention, the power source (10) is positioned in the empty spaces within the ATM (2) and the energy exiting the power source (10) is sent into the cassette outlet (4; belt (7) or band (8) system within the money cassettes (16) by means of the pulsed light system (1) and the disinfection is provided. Therefore, both sides of the paper notes are subjected to the pulsed light system (1). The system can be able to provide disinfection by means of opening every time it detects the paper note by means of a method (sensor, software etc.) which detects the paper bank.

1. System which realizes the inactivation of microorganisms on money by means of the pulsed light technology, characterized in that, it comprises the following steps; transferring the energy received from a power source (10) for generating intensive beam (14) to the light source (13) by taking the same to the inductor (12) after it is stored in a storing capacitor (11), applying the produced UV-C beam with disinfection feature to the target (15) in an intensified manner, performing disinfection of both sides of the money which here and/or passing here by the pulsed light system (1) by means of sending intensive beams (14) which are generated in the money cassettes (16), cassette outlet (4), belt (7) or band (8) system where the light source (13) is placed with the pulsed light system (1) where the power source (10) is placed within the empty spaces in the ATM (2) and energy exits out of the power source (10).

2. System according to claim 1, characterized in that; in the pulsed light system (1), one or more adjustable light source (13) units can be positioned on at least one of the following, the upper and lower selection modules (5, 6), the level of the cassette outlet (4) points, belt system (7) and band (8) system or money inlet (9) sections, besides in the invention since the light source (13) of the pulsed light system (1) can be placed within the money cassettes (16), can be placed in the empty spaces at the side of the upper and lower selection modules (5,6) of the power source (10) and/or within the ATM (2), the light generated by the light source (13) can comprise a wide spectrum of wavelengths from UV to infrared.

3. System according to claim 1, characterized in that; transferring the money that exit from the money cassettes (16) from the cassette outlet (4) to the housing at the beginning of the belt (7) and band (8) system and collecting the same here and then moving the same to the outlet entrance by means of the band system (8) individually or collectively according to the model of the ATM (2) and/or within the money cassettes (16) of the ATM (2), in the path that the money follows as of the exit of the money from the money cassette (16) to its leaving the ATM (2) or applying the combinations thereof are provided with the pulsed light system (1).

4. System according to claim 1, characterized in that; the pulsed light system (1) may provide the disinfection by means of opening each time it detects the paper note with the help of sensors detecting the paper note and software.
